(19) United States
(12) Patent Application Publication
(10) Pub. No.: US 2018/0086683 A1
(43) Pub. Date: Mar. 29, 2018

(54) SMALL SCALE PRODUCTION OF METHOXY COMPOUNDS

(71) Applicant: Richard Sapienza, Phoenix, AZ (US)

(72) Inventor: Richard Sapienza, Phoenix, AZ (US)

(21) Appl. No.: 15/720,781

(22) Filed: Sep. 29, 2017

Related U.S. Application Data
(60) Provisional application No. 62/401,257, filed on Sep. 29, 2016.

(51) Int. Cl. C07C 41/09; C07C 43/04; C07C 29/80; C07C 29/151; C07C 31/04; C07C 67/03; C10J 3/72; B01J 19/00; B01J 8/18

(52) U.S. Cl. CPC C07C 41/09; C07C 43/043; C07C 29/80; C07C 29/1518; C07C 31/04; C10J 2300/1823; C10J 3/721; B01J 19/0066; B01J 8/1818; C10J 2200/09; C07C 67/03

(57) ABSTRACT

A method includes receiving a base raw material at a system. The base raw material is converted to synthesis gas. The synthesis gas is conditioned to remove moisture and carbon dioxide. One or more methoxy compounds are produced from methanol when the methanol is produced from the conditioned synthesis gas at operational temperatures below 205° C.

### FIELD

Embodiments herein relate to the production of methanol, dimethyl ether (DME) and/or other methoxy related products from synthesis gas.

### BACKGROUND

The surging supplies of oil and natural gas are spearheading a revival of chemical and manufacturing investment in regions of the country where oil and gas are being developed. Driven by recent advances in fracking and horizontal drilling, a surge in natural gas production has led to depressed natural gas prices. In turn, this gas boom has created site-specific gas utilization opportunities; where the gas byproduct from oil production is too far from pipelines and thus flared, and the smaller gas volumes have been a limiting factor to economically convert associated gas into valuable products. The World Bank estimates that globally over 150 billion cubic meters (5.3 trillion cubic feet) of natural gas are flared or vented annually, which is equivalent to 25% of the United States' gas consumption or 30% of the European Union's annual gas consumption. Only small-scale and modular systems both on- and offshore can take advantage of this wide range of gas resources that are by themselves too small for larger commercial plants.

Methanol is one of the most versatile compounds developed and is the basis for hundreds of chemicals, thousands of products that touch our daily lives, and because methanol production facilities and principle users span the globe, approximately 80 percent of the world's annual methanol production is transported between continents by transoceanic shipping. Methanol is received and stored at marine terminals, then shipped by truck, rail and barge to chemical production facilities and bulk distributors—where it is stored in tank farms and repackaged into smaller containers. Tanker trucks and trailers complete the distribution network, delivering methanol to the wide range of final users in the methanol value chain.

Although ethane generally is worth much more than an equivalent volume of natural gas, much of the associated ethane produced has one obvious problem: it is not located where the demand is. Midstream shale gas companies want to transport away ethane to stimulate the production of natural gas but there are few regional ethane markets, and often the infrastructure does not yet exist to move ethane to markets outside the region. Methods for the production of methanol by catalytic conversion of synthesis gas containing hydrogen and carbon oxides have long since been known to those skilled in the art. For example, Ullmann's Encyclopedia of Industrial Chemistry, Sixth Edition, 1998 Electronic Release, Chapter “Methanol”, Sub-chapter 5.2 “Synthesis”, describes a method for the production of methanol.

However, while some methanol dehydration reactions are disclosed, existing methods do not disclose reaction schemes that may be employed to effectively provide purified methanol via a single pass from synthesis gas via a low temperature (below 200° C.) and high equilibrium conversion catalyst, which is known to reduce problems in the preparation of products.

### SUMMARY

A method disclosed herein includes receiving a base raw material at a system. The base raw material is converted to synthesis gas. The synthesis gas is conditioned to remove moisture and carbon dioxide. Methanol is produced from the conditioned synthesis gas at operational temperatures below 205° C. The product is free from water. The methanol can be further utilized directly, that is, without separation or isolation, for other products via a methoxy synthesis. One or more methoxy compounds can be produced from the methanol at temperatures below 205° C. The base raw material can be one of a natural gas, a biomass, a carbonaceous base fossil material, a carbonaceous renewable raw material, methane, and ethane.

Production of methanol can include a liquid containing catalyst system operating below 205° C. Converting natural gas to synthesis gas can include converting methane to synthesis gas using a reformer system. The resulting gas can be fed to a continuous flow stirred-tank reactor. The methanol vapor from the reactor can be directed to a reactive distillation tower. Carbon-oxides per pass conversion can be higher than 65% and selectivity to methanol formation is at least 75% with little to no water present.

Dimethyl ether can be produced by integrating a synthesis gas reaction zone and a dehydration zone. Methanol can be introduced into the reactive distillation tower to produce a dimethyl ether top stream and a water bottom stream. Converting natural gas to synthesis gas can include pre-treating the natural gas to remove hydrogen sulfide.

Fatty acid methyl ester can be produced from methanol in the presence of one of an alkali, an alkali-solid, an acidic-solid and an enzyme catalyst, or in the absence of catalyst at atmospheric pressures. Methoxy containing product obtained from methanol, such as methylformate, dimethyl ether or a methyl ester, acid methyl esters, dimethyl ether, methyl t-butyl ether (MTBE), t-amyl methyl ether (TAME), fatty acid methyl esters (FAME) and methyl ethers can be produced. These can be produced as additional products directly from the low temperature methanol synthesis process.

Converting natural gas to synthesis gas can be carried out by a method selected from the group consisting of oxygen transport membrane reforming, steam reforming, partial oxidation, autothermal reforming, dry reforming, gasification and combinations thereof. Converting natural gas to synthesis gas can include producing at least 50% methyl formate.

A method for the production of methanol formate from the synthesis gas can include a liquid catalyst system operating below 205° C. A methyl formate production system can be integrated for continuous production of methoxy containing product obtained from methyl formate. Carbon-oxides per pass conversion can be higher than 65% and selectivity to methanol formation is at least 75%.

The low temperature methyl formate production system can be integrated for continuous production of methoxy containing product obtained from methyl formate.

These and other features of the systems and methods of the subject disclosure will become more readily apparent to those skilled in the art from the following detailed description of the preferred embodiments taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, preferred embodiments thereof will be described in detail herein below with reference to certain figures.

FIG. 1 is a schematic of an exemplary process for converting natural gas to methanol and DME in accordance with an embodiment herein.

FIG. 2 is a schematic of an exemplary process for converting synthesis gas to methanol and DME according to an embodiment herein.

These and other aspects of the subject disclosure will become more readily apparent to those having ordinary skill in the art from the following detailed description of the invention taken in conjunction with the drawings.

### DETAILED DESCRIPTION

Reference will now be made to the drawings wherein like reference numerals identify similar structural features or aspects of the subject disclosure.

The methanol production process generally involves directing a compressed synthesis gas comprising hydrogen, carbon monoxide and carbon dioxide to a methanol reactor containing one or more beds of a methanol synthesis catalyst, such as copper and zinc oxide catalyst, at elevated temperature with the preferred range of 220° C. to 260° C. and pressure in the range of 2 megapascal (MPa) to 10 MPa. The carbon monoxide and carbon dioxide in the synthesis gas react with the hydrogen to form methanol across the catalyst. The methanol synthesis process is usually operated in a loop where a portion of the compressed synthesis gas is converted to methanol in each pass through the methanol reactor. Methanol product is recovered by cooling the methanol product gas stream to a temperature below the dew point of the methanol such that crude methanol and water condense out, with the remaining gas being recycled through the methanol reactor. The crude methanol and water produced in the methanol reactor are typically directed to a let-down or “flash vessel.” Since most crude methanol contains a large range of impurities, the crude methanol must be purified so as to remove water and such impurities to produce methanol of chemical grade quality. The preferred technique used for methanol purification is a distillation process.

Historically, gas to chemical production at gas well sites or at other remote synthesis gas locations has been prohibitively expensive as compared with refinery sited facilities. Most existing natural gas to product efforts are modular plants that can only economically convert gas in the 1,000 to 15,000 barrels/day and cost about $200,000 for every barrel/day capacity. Further a 1,000 barrel per day plant requires the availability of approximately 100 billion cubic feet of natural gas over the 25-year usable plant life. However, over 50% of the world's gas reserves are relatively small fields that would be inadequate for these production capacities. Therefore, improved gas-to-chemical production technologies that may be viable at production capacities below 1,000 bpd are needed for these locations.

The use of biomass to obtain liquid fuels has become attractive. Biomass can be converted to synthesis gas to make liquid fuel; animal oil or vegetable oil can be converted to biodesel due to their compatibility with current automobiles and petrol supply chains. However, the profitability of biofuels depends heavily on the economy of the byproducts. For some time, glycerol has been a valuable byproduct in the biodiesel industry when using vegetable or animal fats/oils. However, the increase in the production of biodiesel (a.k.a., FAME), a methoxy functional acid, results in an excess of glycerol with a limited market, reducing the price. Under these expected revenues from glycerol, there is a need to reduce the production cost of FAME biodiesel and an integrated process to generate methanol for methoxy functionality will be ideal and competitive with existing technologies. Furthermore, the production of methanol from glycerol is also meant to reduce the dependency of biodiesel on fossil fuels. See, for example, U.S. Pat. No. 7,857,869.

Methyl tertiary butyl ether (MTBE) and other methoxy related products are in high demand. For example, in the 1990s, large amounts of methanol were used in the United States to produce the gasoline additive MTBE the methyl ester of t-butanol. While MTBE is no longer marketed in the U.S., it is still widely used in the other parts of the world as an octane booster/additive in reformulated gasoline.

Embodiments herein provide advances in DME and methoxy functionality plant operations, including methanol itself and, more particularly, advances in the integration of the small scale, low temperature production of methanol from synthesis gas with the DME synthesis or the front-end of the plant.

Embodiments herein provide methods and systems for producing DME and methoxy functionality via a process that directly integrates a low temperature process for methanol from synthesis gas reaction zone, a dehydration reaction zone, and optionally a separation zone, thus optimizing the process based on economic and engineering considerations.

Embodiments herein relate to a process for preparing DME and related methoxy functionality (e.g., methyl esters) products from methanol. Embodiments herein also relate to the preparation of conversion products thus obtained using natural gas reserves that are economically stranded or flared. Embodiments herein further relate to the preparation of conversion products thus obtained using any source of synthesis gas that can be converted to methanol. The process allows for producing solely methanol. The process allows for producing DME and methoxy functionality from methanol by: a reaction step for continuously allowing methanol to be fed and reacted in the necessary conversion reaction zone of the desired products system. It therefore also allows the continuous production of these chemicals from a back-end synthesis gas basis from any synthesis gas source and not from methanol made in a separate facility or location. The disclosed processes generally function at lower temperatures and at lower pressures than conventional processes so as to produce higher DME and methoxy functionality yields than do conventional processes.

In the larger economic picture, this type capability can be the key factor that enables the construction of upstream projects that would otherwise be cancelled because of poor results derived from economic models. For example, some shale gas discoveries are being hampered by high development costs which result in marginal economics due to gas prices that are often low.

Moreover, existing methods and systems suffer from poor heat control (transfer), short lifetimes (due to heat) and the need for distillation or another separation zone. Embodiments herein take advantage of a very low temperature methanol synthesis condition which highly favors methanol formation from synthesis gas. Various embodiments herein prefer the use of a liquid containing catalyst system, whether homogenous or slurry, that allows heat transfer to be decoupled from kinetics and offers a method to overcome the most important technological and non-technological barriers to the implementation of under 1000 barrels/day chemical production. The low temperature operation gives high equilibrium yields and small sacrificial energy needs and with high selectivity and little to no distillation is needed.

The low temperature system disclosed herein can convert synthesis gas nearly completely to methanol in a single pass through the synthesis reactor. This improvement is a function of equilibrium. The high conversion eliminates recycle. High conversion of syngas in addition to the low vapor pressure of methanol causes the methanol product to reflux within the reactor providing a very pure product not requiring distillation, and thereby permits the use of this product methanol at reaction temperature and pressure to any downstream process, such as DME.

The systems disclosed herein are smaller-sized, well under 1,000 barrel per day plants, which make for a logical and commercially viable solution for 50% or more of stranded and associated gas with no alternative economical outlet. In the larger economic picture, this type capability can be the key factor that enables the construction of upstream projects that would otherwise be cancelled because of poor results derived from economic models. For example, some shale gas discoveries are being hampered by high development costs, which result in marginal economics due to gas prices that are often low. These projects can be enhanced by converting the gas to valuable chemicals in the disclosure process. The goal is to create systems both on and offshore to take advantage of a wide range of gas resources that are by themselves too small for larger multibillion dollar chemical or fuel plants.

The process according to various embodiments herein possesses an increased capacity of DME formation based on the amount of methanol converted from synthesis gas derived from base fossil raw materials. The methanol production process may employ an oxygen transport membrane based reforming system, such as that described in U.S. Pat. No. 9,115,045, or synthesis gas generation systems (e.g., steam methane reforming, Autothermal reforming, Catalytic Partial Oxidation, Bi-reforming, CO₂ reforming etc., and their combinations) in accordance with the embodiments herein.

As used herein, base raw materials may refer to, for example, natural gas, brown coal, hard coal and mineral oil, and renewable raw materials which are obtained from biomass, e.g., from vegetable and animal material which is grown (in the present and/or will be grown in the future).

The production of DME is of considerable commercial use, because the nature of DME is similar to that of liquidated petroleum gas, DME can be added into liquidation petroleum gas for replacement. Hence, DME is deemed a new energy carrier in the 21^{st} century. Furthermore, the combustion of DME is cleaner than that of diesel. Therefore, DME is more environmentally friendly than fossil fuel.

Embodiments herein may also have a significant niche to fill in the emerging energy picture by increasing opportunities for biomass, waste and coal conversion via syngas. Most of these projects are considered longer term development efforts because these projects are more complex due to the additional processing steps, however this invention provides a low-cost, flexible fuel step for these systems to be introduced which no other chemical process can.

The kinetics and mechanism for methanol synthesis from synthesis gas have been studied extensively. (Strelzoff, S., “Methanol: Its Technology and Economics,” Methanol Technology and Economics, Chemical Engineering Progress Symposium Series, No. 98, Vo.66, AIChE, 1970.) The equilibrium conversion of synthesis gas to a liquid fuel is higher at lower temperatures. Therefore, the key consideration in designing a conventional methanol synthesis reactor and a slurry bubble column reactor is removal of the heat of reaction and maintaining operation in a specific temperature range, namely 428-518° F. (220-270° C.) for methanol. Isothermal operation at about 482° F. is the optimum condition for methanol. Methanol is presently manufactured from synthesis gas using various supported Cu/ZnO heterogeneous catalysts at 250° C.-300° C. that poses thermodynamic limitations and together with poor process heat control allows >20% per pass gas conversion during the reaction. This low carbon oxide per-pass conversion has been attributed to the thermodynamic limitation of the highly exothermic reaction. Consequently, the recycling of unreacted carbon oxides is necessary to enhance their conversion, leading to a higher production cost associated with a more sophisticated process design.

Embodiments herein are directed to processes for the preparation of methanol at temperatures below 205° C., having process carbon oxides per-pass conversion equal to or higher than 65%, and selectivity to methanol formation equal to or higher than 75%. Example systems include a binary catalyst based on the combination of a homogeneous potassium formate catalyst and a solid copper-magnesia catalyst in alcohol solvents was used for methanol synthesis starting from syngas at low temperature. The processes are operated at a pressure from 0.5 to 10 Mpa abs. and up to 30 megapascal absolute (Mpa. Abs.).

DME is commercially made by dehydrating methanol all over the world. In a commercial process, there are two distinct processes, methanol synthesis with its purification and DME synthesis for which two devices are used, i.e., a reactor and a DME separation and purification device. The DME separation and purification device is a distillation tower or packing tower in most cases. Methanol is usually sourced elsewhere and brought to the DME commercial site. Pure methanol is pumped, heated, vaporized, and often superheated in a heat exchanger, and then sent to the reactor in which dimethyl ether (DME) is formed.

A process that integrates methanol from synthesis gas reaction zone, and a dehydration zone for making DME by reactive distillation is preferred. Still more preferred is a method for making DME by reactive distillation of methanol in a single piece of equipment, such as reactive distillation tower, for example, but not limited to, one such as that described in U.S. Pat. No. 8,816,134.

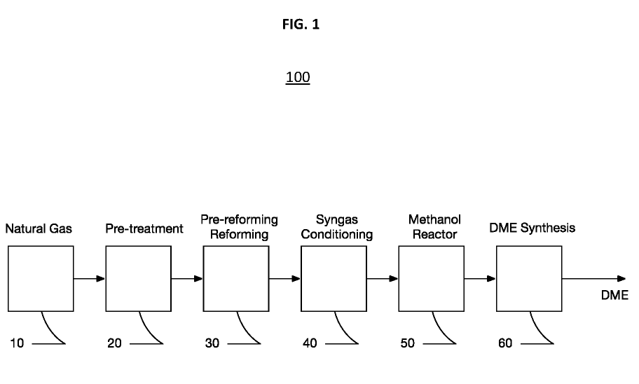

Referring to the figures, FIG. 1 shows an illustrative process 100 for producing methanol and DME from natural gas. Natural gas is received at block 10, the natural gas then undergoes pre-treatment at block 20 and undergoes pre-reforming/reforming at block 30. Syngas conditioning occurs at block 40. The syngas is the processed at block 50 to produce methanol. Methanol is dehydrated in the reactor to provide DME and water. At block 60, the DME is collected from a stream produced after the reaction and purified in the dimethyl ether separation and purification device. Methanol is introduced into the reactive distillation tower so that the methanol travels from the reaction zone toward the bottom for contact with the catalyst to provide a top stream and a bottom stream. The top stream includes DME. The bottom stream includes water and remaining portion of the methanol. Finally, the DME is collected in the top of the reactive distillation tower. The purity of the collected dimethyl ether is higher than 99%.

The reactive distillation process is based on chemical equilibrium and Le Chatelier's principle. For chemical reactions that are limited by chemical equilibrium, reactants are instantly separated from products. A product with a higher relative volatility is separated from a reactant with a lower relative volatility in a distillation tower. The product with the higher relative volatility is removed from the distillation tower to facilitate further production of the product and reduce risks of side reactions. For example, in embodiments herein, the reaction taking place is two methanol molecules forming DME: 2CH₃OH→CH₃OCH₃+H₂O. In this instance, the product with the higher volatility would be the DME.

Therefore, coupling methanol synthesis to its reactive distillation to DME is commercially beneficial. Embodiments herein result in the formation of DME from all carbonaceous base materials via synthesis gas.

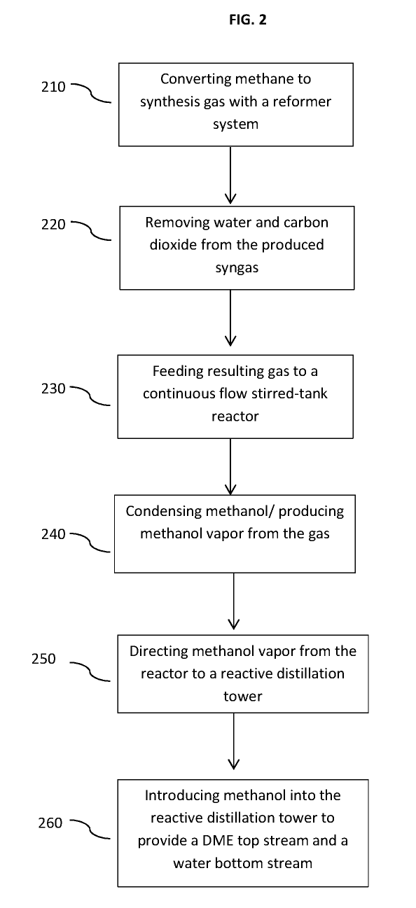

FIG. 2 shows an exemplary process further detailing production of methanol and DME according to an embodiment herein. At block 210, methane is converted to synthesis gas with a reformer system (e.g., NOVOROCS Onsite Flare Gas Unit). At block 220, water and carbon dioxide is removed from the produced syngas to 1 ppm and 10 ppm using dual molecular sieve Temperature Swing Adsorption (TSA) units. At block 230, the resulting gas is feed to a continuous flow stirred-tank reactor (CSTR) containing a formula which does not form nickel carbonyl. At block 240, high activity of 7 grams of methanol per hour per cubic centimeter of catalyst solution can be achieved and methanol condensed can be as pure as 99.5%. Simple bubble columns gave 3 gm/cc/hr. The preferred slug flow units achieved 7-9 gm/cc/hr. At 3 gram/cc/hr a single reactor of 5.75 foot diameter and 40 foot height, including entrainment removal space, would produce 1,000 metric tons per day of methanol and the capital charges of the reactor investment will be 0.003 cents per round of methanol.

At block 250, the methanol vapor from the reactor at 6-7bars was directed to a reactive distillation tower. The pressure could be obtained from the reaction of the synthesis gas to methanol. The top is retained at 25° C. to 40° C. while the bottom of the reactive distillation tower is retained at 84.60° C. to 170° C. At block 260, the methanol is introduced into the reactive distillation tower so that the methanol travels from the reaction zone toward the bottom for contact with the acidic ion exchange resin catalyst to provide a top stream and a bottom stream. The top stream includes DME. The bottom stream includes water. The dimethyl ether is collected in the top of the tower. The purity of the collected dimethyl ether is 99%.

Existing systems need to distill the methanol before it can be used to make additional products. Methods and systems described herein allow the methanol to flow directly into the dehydration reactor to produce DME. Embodiments herein provide a method for making dimethyl ether by dehydrating methanol while consuming a reduced amount of energy. The dehydrate zone can also be used for the production of other ethers when the appropriate co-reactant is introduced into the reaction zone. One example of a commercial product is a methyl tertbutyl ether (MTBE). The process comprises continuously feeding t-butyl alcohol, and/or isobutylene and methanol into a packed solid-acid catalyst bed, in a reactor-separator distillation column in the presence of the solid-acid catalyst, whereby a product of substantially pure MTBE is separated from the reaction mixture.

The process disclosed herein provide for the continuous production via esterification of methyl esters. Esterification is the general name for a chemical reaction in which two reactants (typically an alcohol and an acid) form an ester as the reaction product. One commercial example is the fatty acid methyl esters (FAME), in particular for diesel combustion engines, from vegetable and/or animal fats and/or oils produced through fermentation of oil-bearing microbes and/or recombinant deoxyribonucleic acid (DNA) techniques are used to produce oleaginous recombinant cells that produce triglyceride oils having desired fatty acid profiles and region specific or stereospecific profiles by transesterification of the triglycerides contained in the fats and oils with methanol in the presence of an alkali catalyst, an alkali solid catalyst, an acidic solid catalyst or an enzyme catalyst or in the absence of catalyst and at atmospheric pressures. Existing methods of producing fatty acid alkyl esters through a combination of esterification and transesterification processes catalyzed by acid and alkali may be used, for example, U.S. Pat. No. 6,399,800 and U.S. Pat. No. 7,109,363. The purification of methyl ester and utilization of crude glycerol layer may also be performed using standard methods. It is stated therein that mono-and diglyceride impurities in the glycerol layer can be converted into the desired methyl ester through reaction with additional amounts of methanol. It is noted here that methyl formate is the methyl ester of formic acid (methoxy formic acid).

Small scale production of methanol derived chemicals such as DME and other ester derivatives such as FAME biodiesel (e.g. the autocatalytic methoxy esterification of fatty acids) via most conventional processes have been limited by economic and environmental constraints. As can be appreciated, conventional methods of producing DME and methoxy functionality are expensive and involve complex installations. An objective of this disclosure is to overcome the complexity and expense of such installations at the small scale of stranded and associated gas reserves. This invention discloses converting natural gas into fuels or chemicals on a small scale, and doing that in a distributed manner as opposed to a centralized manner, since the technology and business model that have been typically used don't work.

By eliminating intermediate condensation steps and utilizing a highly integrated design, this disclosure also applies to multi-step preparations such as a process for producing ethanol from synthesis gas by reacting the synthesis gas to provide methanol, which then is subjected to dehydration to produce at least one ether, such as dimethyl ether, then is subjected to carbonylation with carbon monoxide from the synthesis gas to provide at least one acetate, such as methyl acetate. The acetate then is subjected to hydrogenolysis to produce ethanol, or the conversion of methanol to DME and subsequently to hydrocarbons.

One advantage of a DME preparation process based on the raw material methanol is that the methanol can be obtained via synthesis gas (gas mixtures of carbon monoxide and molecular hydrogen) in principle from all carbonaceous base fossil materials and all carbonaceous renewable raw materials. As in the case of methane, the molecular hydrogen required may already be present in the carbon carrier, for example, using a standard process for obtaining methane from biogas or biomass. For example, a process for obtaining methane from biogas or biomass is described in DE-A 102008060310 and EP-A 2220004. An alternative hydrogen source is water, from which molecular hydrogen can be obtained, for example, by means of electrolysis. The oxygen source is generally air. A suitable renewable carbonaceous raw material for synthesis gas production is, for example, lignocellulose. It is also possible to obtain synthesis gas by coupling the pyrolysis of biomass directly with steam reforming.

Thus embodiments herein overcome the following key challenges to stranded natural gas coal-bed methane and biogas development:
a. Complexity—technical level of difficulty is not high
b. Capital Cost—No high barrier to entry; NOT difficult to finance
c. Risk—offers options for volatile gas/oil prices; high methane cost; complexity.

The following examples will serve to further illustrate processes and some advantages of embodiments herein.

### EXAMPLE 1

Conventional On-Site Production of DME and Methoxy Functions from Natural Gas.

250,000 SCFD of natural gas is converted to 2000 gallons of methanol and then the methanol is converted to 1000 gallons of DME at a remote location near the production site of natural gas. The capital cost for the project will be at least 9.5 million dollars.

### EXAMPLE 2

Integrated process for the Production of DME and Methoxy Functions from Natural gas.

With reference to FIG. 1 above, 250,000 SCFD of natural gas at production site 10 undergoes pretreatment 20 to remove H₂S and it will be converted to synthesis gas through pre-reforming and reforming steps 30. Furthermore, the resulting syngas is conditioned to remove CO₂ and moisture in 40. The conditioned syngas is then introduced to methanol reactor 50 and methanol product is directly introduced into the reactive distillation unit 60 to produce DME. The capital cost for this project will be around 3.3 million dollars.

The examples demonstrate that using an integrated approach to produce DME and methoxy functions from natural gas the required capital for the project can be significantly reduced as much as 60%. Using this integrated approach, the product from methanol (methoxy) synthesis has been integrated with the syngas to methanol synthesis, providing for the elimination of the condensation and distillation steps of conventional methanol synthesis, potential need for methanol liquid transport, and the reheating of the methanol for the subsequent methoxy reaction. Current technologies require significant capital investment for processes under 1000 b/d chemical production. Embodiments herein allow co-production of DME and methoxy functions at much lower cost.

Methanol is one of the seven highest volume commodity petrochemicals, with a consumption of more than 40 million ton per year. Industrially it is widely applied and there are various technologies available for licensing. The normal criteria for the selection of technology are capital cost and plant efficiency. A methanol plant with natural gas feed can be divided into 5 main sections. In the first part of the plant oxygen is separated from air [or a steam plant used] 2) natural gas is converted into synthesis gas with oxygen/steam. 3) The synthesis gas reacts to produce methanol in the second section 4) unutilized synthesis gas is recycled to reactor, and 5) methanol is purified to the desired purity in the tail-end of the plant. The capital cost of large scale methanol plants is substantial. The synthesis gas production including compression and oxygen production when required may account for 60% or more of the investment. With current commercial systems methanol production of up to around 7,000 TPD is a lower limit and cost about $90,000 for every b/d capacity.

TABLE 1
Comparison of conventional methanol processes to exemplary
processes according to embodiments herein.

| | | | Syngas | |
|---|---|---|---|---|
| | | Temperature | Pressure | % Methanol |
| Process | | (° F.) | (psi) | Conversion |
| Conventional | Lurgi HP | 752 | 5300 | 48 |
| Process | Lurgi LP | 491 | 966 | 61 |
| | ICI LP | 527 | 1131 | 55 |
| Exemplary | Low temperature | 230 | 206 | 93 |
| Processes of | Liquid Methanol | | | |
| Present | Low temperature | 230 | 735 | 97 |
| invention | Liquid Methanol | | | |
| | Low temperature | 230 | 206 | 87 |
| | Liquid Methanol | | | |
| | with 40% N2 in | | | |
| | Syngas Feed | | | |

Modularization for smaller scale F-T plants are limited to 1000-5000 barrels/day and cost about $200,000 for every b/d capacity.

TABLE 2
Comparison of Exemplary Embodiment and Conventional
Methanol Synthesis

| | Exemplary | Conventional |
|---|---|---|
| | Embodiment | Methods |
| Reactor temperature(° C.) | 110 | 265 |
| Reactor pressure (psia) | 150 | 750 |
| Equilibrium CO conversion (%) | 94 | 61 |
| Operating CO conversion (%) | 90 | 16 |
| Volume of gas recycle (mols CO/mol prod.) | 0 | 5.25 |
| Reactor feed (mols CO/mol prod.) | 0.94 | 6.25 |
| Overhead gas cooling duty (Btu/mol prod.) | 4,100 | 71,000 |

Advantageously, embodiments herein address critical themes of specific importance:
(a) Reduces complexity. Robust technology can be optimized, but it is difficult to make already complex technology robust.
(b) Reduces capital by innovation. To stop increasing the size of old technology and develop new technology based on better understanding of the process science.
(c) Tackles the difficult problems. Some big issues:
   No Air separation. In a convention gas to liquids facility, ˜30% of the capital cost is due to cryogenic air separation and utilities. Synthesis gas production including compression and oxygen production when required may account for 60% or more of the methanol investment
   Improve reaction engineering for better once through yields. Conventional methanol require recycling of feed syngas an expensive and complex proposition.
   The removal of process heat that can produce hot spots and severely shorten catalyst life in both the exothermic methanol synthesis.

The methods and systems of the present disclosure, as described above and shown in the drawings, provide for removal of high freeze point hydrocarbons at higher pressure than conventional systems. While the apparatus and methods of the subject disclosure have been shown and described with reference to preferred embodiments, those skilled in the art will readily appreciate that changes and/or modifications may be made thereto without departing from the scope of the subject disclosure.

What is claimed is:

1. A method for producing methoxy compounds from a base raw material, comprising:
receiving the base raw material at a reformer system;
converting the received base raw material to synthesis gas at the reformer system;
conditioning the synthesis gas to remove moisture and carbon dioxide;
producing methanol from the conditioned synthesis gas at operational temperatures below 205° C.; and
producing one or more methoxy compounds from the methanol at operational temperatures below 205° C.

2. The method of claim 1, wherein producing methanol includes a liquid containing catalyst system operating below 205° C.

3. The method of claim 1, wherein converting natural gas to synthesis gas includes converting methane to synthesis gas using a flare gas unit.

4. The method of claim 3, further comprising feeding resulting gas to a continuous flow stirred-tank reactor.

5. The method of claim 4, further comprising directing methanol vapor from the reactor to a reactive distillation tower.

6. The method of claim 5, further comprising producing the one or more methoxy compounds by integrating a synthesis gas reaction zone and a dehydration zone.

7. The method of claim 6, further comprising introducing methanol into the reactive distillation tower to produce a dimethyl ether top stream and a water bottom stream.

8. The method of claim 1, wherein converting base raw material to synthesis gas includes pre-treating the natural gas to remove hydrogen sulfide.

9. The method of claim 1, further comprising production of fatty acid methyl ester from the methanol in the presence of one of an alkali, an alkali-solid, an acidic-solid and an enzyme catalyst.

10. The method of claim 1, further comprising production of fatty acid methyl ester from the methanol in the absence of catalyst at atmospheric pressures.

11. The method of claim 1, wherein the one or more methoxy compounds include methylformate, dimethyl ether, and methyl ester.

12. The method of claim 11, wherein the one or more methoxy compounds further include acid methyl esters.

13. The method of claim 11, wherein the methoxy containing product further including dimethyl ether, methyl t-butyl ether (MTBE), t-amyl methyl ether (TAME) and methyl ethers.

14. The method of claim 1, wherein converting the base raw material to synthesis gas is carried out by a method selected from the group consisting of oxygen transport membrane reforming, steam reforming, partial oxidation, autothermal reforming, dry reforming, gasification and combinations thereof.

15. The method of claim 1, wherein converting the base raw material to synthesis gas includes producing at least 50% methyl formate.

16. The method of claim 15, further comprising integrating a methyl formate production system for continuous production of methoxy containing product obtained from methyl formate.

17. The method of claim 1, wherein carbon-oxides per pass conversion is higher than 65% and selectivity to methanol formation is at least 75%.

18. The method of claim 1, wherein the base raw material is one of a natural gas, a biomass, a carbonaceous base fossil material, a carbonaceous renewable raw material, methane, and ethane.

19. The method of claim 18, wherein the natural gas is obtained from a stranded natural gas reserve.

20. An apparatus for producing methoxy compounds from natural gas received from a natural gas reserve, comprising:
a drilling operation for recovering a natural gas comprising methane;
said drilling operation operatively connected to a reformer system configured to convert the natural gas to a synthesis gas;
a temperature swing adsorption unit for receiving said synthesis gas from said reformer system and configured to condition the synthesis gas to remove moisture and carbon dioxide to produce a conditioned synthesis gas;
a continuous flow stir-tank reactor configured to receive the conditioned synthesis gas from said adsorption unit and to produce methanol from the conditioned synthesis gas; and
a reactive distillation tower configured to receive said methanol from said continuous flow stir-tank reactor to produce methoxy compounds from the methanol at operational temperatures below 205° C.

21. In a process for gas drilling comprising flaring a methane containing gas, the improvement comprising employing at least a portion of the methane-containing gas to produce methoxy compounds via production of methanol from synthesis gas at operational temperatures below 205° C., the process being performed in an apparatus at the same location as said gas drilling process.
